(19) United States
(12) Patent Application Publication
(10) Pub. No.: US 2009/0018430 A1
(43) Pub. Date: Jan. 15, 2009

(54) METHOD FOR PRODUCING A MEDICAL IMAGE AND AN IMAGING DEVICE

(76) Inventor: Eike Rietzel, Darmstadt (DE)

Correspondence Address: Brinks-Hofer Gilson & Lione, P.O. Box 10395, Chicago, IL, 60610, US

(21) Appl. No.: 12/167,807

(22) Filed: Jul. 3, 2008

(30) Foreign Application Priority Data
Jul. 12, 2007 (DE) DE 102007032530.6

(51) Int. Cl. A61B 5/05; A61B 5/02

(52) U.S. Cl. 600/407; 600/587

(57) ABSTRACT

A method for examining a patient in a medical imaging device is provided. The method includes measuring a pressure inside the body of the patient over a period of time during the examination, recording measurement data originating in the patient while the examination is in progress, reconstructing the medical image from the recorded measurement data, wherein the recording of the measurement data is controlled by the measured pressure and/or wherein the measured pressure is taken into account while the medical image is being reconstructed from the recorded measurement data.

This application claims the benefit of DE 10 2007 032 530.6 filed Jul. 12, 2007, which is hereby incorporated by reference.

### BACKGROUND

The present embodiments relate to producing a medical image.

Medical imaging methods are used to obtain informative images, which allow inferences to be made about anatomical and/or functional conditions inside a body being examined. X-ray-based methods include, for example, radiography, computer tomography (“CT”), and “cone-beam” CT. Methods based on nuclear magnetic resonance include, for example, magnetic resonance imaging or magnetic resonance (“MR”) spectroscopy. Nuclear medical imaging methods measure radioactive decay caused by injected radio pharmaceuticals. Nuclear medical imaging methods may include, for example, PET (“positron emission tomography”) imaging and SPECT (“single-photon-emission computed tomography”) imaging. There are further known imaging methods besides these.

The imaging methods need a relatively long time to register the measurement data required to be recorded, with several minutes not being uncommon. Movements inside the part of the body being examined can occur while images are being recorded, and this can result in substantial artifacts or a lack of clarity in the images reconstructed from the image data.

Various methods are aimed at partially resolving the time needed to register the measurement data. For example the respiratory cycle can be recorded and then used for controlling the recording or reconstruction of the image data. Respiratory movements can be prevented from leading to poor image quality. This is similarly applicable to beating-heart movements, which can be monitored by recording an ECG signal.

### SUMMARY AND DESCRIPTION

The present embodiments may obviate one or more of the problems or limitations inherent in the related art. For example, in one embodiment, artifacts or a lack of clarity due to movements to be minimized in recorded images are minimized.

In one embodiment, a method for examining a patient's body in a medical imaging device for producing a medical image is provided. The method includes measuring a pressure inside the patient's body over a period of time during the examination, recording measurement data originating in the patient while the examination is in progress, and reconstructing the medical image from the recorded measurement data. The recording of the measurement data may be controlled with the measured pressure. The medical image may be reconstructed from the recorded measurement data using the measured pressure.

Pressure variations may occur inside a body. The pressure variations may shift the position of an organ or part of the body being examined. Since the pressure inside the patient's body is measured while the examination is in progress, the pressure variations may be detected and enable an indirect inference to be made about the position of the organ being examined. For example, an increased pressure can indicate that a specific organ has moved. When the pressure has returned to normal, pressure can indicate that the organ has returned to its original position. The measured pressure is then used to at least partially control the recording of the measurement data so that any changes in the position of the organ or part of the body being examined may be taken into account while the measurement data is being recorded. The quality of a then reconstructed image may be enhanced.

It is alternatively or additionally possible to use the measured pressure while the medical image is being reconstructed from the recorded measurement data. Any changes in the position of the organ or part of the body being examined may be taken into account while the measurement data is being recorded.

The method enables the examining of organs whose position has been determined by pressure variations inside the body to be performed simply and efficiently. For example, examining abdominal organs whose position often depends on intestinal gases that are located in the abdomen and moving. Inferences may be made about the position of the abdominal organs by measuring the pressure inside the body.

In one embodiment, the recording of the measurement data may be controlled in such a way that the measured pressure is evaluated while the examination is in progress and that the recording of the measurement data will be temporarily interrupted if the measured pressure is outside a tolerance range or exceeds a threshold value. The change in the position of the organ or part of the body being examined is largely reversible once the pressure has returned to normal. The recording of measurement data may be resumed as soon as the measured pressure is within the tolerance range or below the threshold value. The exemplary embodiment may be advantageous for the recording of the measurement data is accompanied by dosing the patient with radiation, for example, if X-rays are used for examining the patient as in the case of, for example, cone-beam CT.

In one embodiment, while the medical image is being reconstructed, for that part of the recorded measurement data in the case of which the measured pressure was outside a tolerance range or exceeded a threshold value during recording to be discarded. The recording of the measurement data may not accompanied by additionally dosing the patient with radiation that can be temporarily deactivated, or if no moving of the detectors takes place for recording the measurement data, as, for example, during a nuclear medical examination (PET, for instance). During a nuclear medical examination, a tracer, for example, will already have been introduced into a body so that interrupting the examination will not lead to a reduction in the radiation dose. In this way the recording of the measurement data may be performed without the need to additionally control recording. The two embodiment variants may be combined.

Instead of discarding the measurement data, it is possible also for specific measurement data in the case of which the measured pressure reached non-desired values during recording to be weighted during image reconstruction with other weighting factors. It is, though, also possible during the evaluation to sort the measurement data according to pressure values or pressure conditions in order to obtain a plurality of reconstructions under different pressure conditions and movement conditions. This will be expedient in particular in the case of periodic movements or in the case of repeatedly occurring different conditions or in the case of measurements over a lengthy period. If during a PET examination, for example, a gas pressure in the rectum increases for tens of seconds or even for minutes and is virtually constant, an image characterizing a further movement condition can be constructed therewith during reconstruction.

In one embodiment, the medical imaging device may be a nuclear medical imaging device, a magnetic resonance imaging device, or a computer tomography imaging device. The method may be used in association with imaging methods such as, for example, PET or SPECT imaging, MR spectroscopy, or cone-beam computer tomography.

In one embodiment, the pressure is measured in a hollow organ of the body. Pressure variations may be measured in organs frequently subject to pressure variations, such as hollow organs. The pressure does not, though, of necessity have to be measured in a hollow organ; for example, the pressure inside the abdominal wall or inside other tissues/organs that are not hollow can also be measured. The pressure can therein be measured particularly in the intestine.

In one embodiment, the measured pressure inside the body is a gas pressure. The pressure variations may be measured that are produced particularly by intestinal gases or the movement of masses of intestinal gas. Imaging-based examination methods that image organs in the intestinal region may be improved. For example, a nuclear medical examination performed on the prostate, in the lower abdomen, or on other organs within the abdomen may be improved. The pressure can, though also be measured in other hollow organs such as, for example, the bladder, lungs, stomach, and elsewhere.

In one embodiment, an imaging device for examining a patient includes a sensor, a recording unit, and a processing unit. The sensor is operable to measure a pressure, such as a gas pressure, inside the body of a patient while the measurement data is being recorded. The recording unit is operable to record measurement data originating in the patient. The processing unit is operable to process the recorded measurement data, so that a medical image may be obtained from the measurement data. The imaging device is operable to implement a method as discussed above.

The sensor may measure pressure in a hollow organ of the body, such as the intestine. For example, the sensor may be embodied in such a way that the sensor may be inserted rectally. The access path allows the sensor to be applied to a patient without any problems.

The sensor, which measures the pressure inside the body, may measure gas pressures. The sensor may be used to detect, for example, pressure variations in the intestine due to masses of intestinal gas.

The sensor may be disposed on a valve mechanism for releasing excess pressure. For example, excess pressure forming in the intestine due to masses of intestinal gas may be released rectally via the valve mechanism. The pressure conditions in the intestine may return to normal faster so that, for example, the recording of the measurement data can be resumed faster. The valve may remain open until the excess pressure in the intestine has been released.

The sensor may be disposed on an inflatable balloon. An inflatable rectal balloon will, for example, additionally support the immobilizing of organs, such as the prostate.

In an embodiment variant a sensor signal generated by the sensor, for example, can be evaluated by an evaluation unit. The evaluation unit may identify, for example, a departure of the sensor signal from a tolerance range or the exceeding of a threshold value. While this condition persists, the recording unit for recording measurement data will be controlled so that the recording is interrupted. The measurement data recorded under this condition may be discarded during the reconstruction of an image.

### BRIEF DESCRIPTION OF THE DRAWINGS

FIG. 1 shows one embodiment of a schematic sagittal section through a human lower abdomen,

FIG. 2 illustrates one embodiment of an imaging device, and

FIG. 3 illustrates one embodiment of an imaging device method.

### DETAILED DESCRIPTION

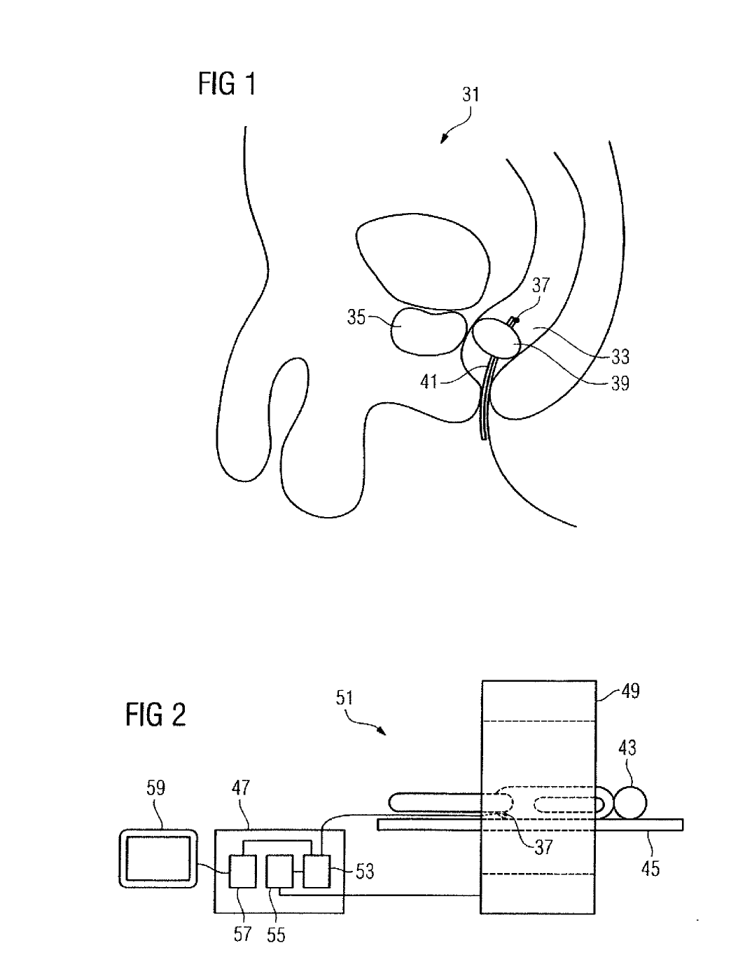

FIG. 1 shows a schematic sagittal section 31 through the lower abdomen of a male body. Indicated schematically are only the external contours and the contours of the organs necessary for the explanations below. In the lower abdomen near the rectum 33 is the prostate 35. The prostate 35 is an organ that may be affected by tumors; and therefore, may be imaged with a medical imaging device in order to make a diagnosis or determine the advance of a disease. Nuclear medical methods are used for imaging, for example, PET imaging or SPECT imaging, possibly in combination with CT, and other imaging methods such as MR or cone-beam CT imaging.

The prostate 35 may be subject to movements inside the body (internal movements), while image data is being recorded. The internal movements are due predominantly to a formation of gas and/or a shifting of masses of gas in the adjacent intestine or adjacent rectum 33. The internal movements may result in unclear images or in artifacts in the images.

A sensor 37 may measure pressure inside an intestine. The sensor 37 may be rectally inserted into the rectum 33. The sensor 37 may be disposed on a rectal balloon 39 that may be used for immobilizing the prostate 35. The rectal balloon 39 may be inserted into the rectum 33 in a folded condition. The prostate 35 may be mechanically fixed by inflating the rectal balloon 33.

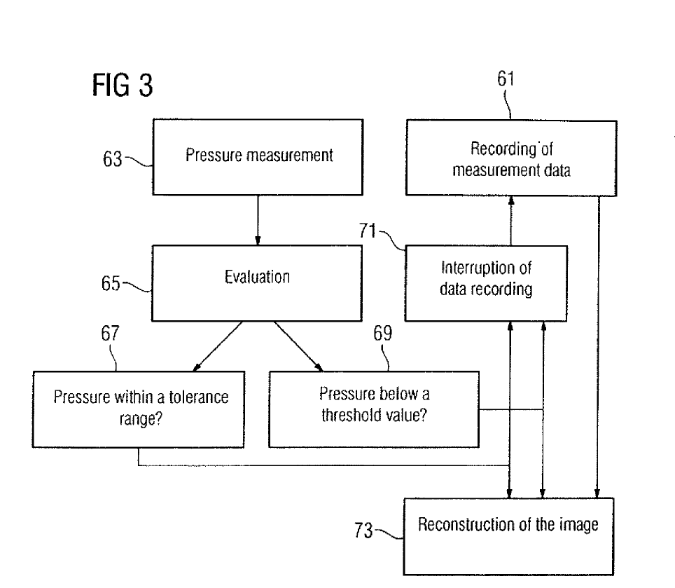

An increase in pressure in the rectum 33 or intestine due to masses of gas may shift the position of the prostate 35. The pressure variations may be detected by the sensor 37 so that the medical imaging device can be controlled based on them, as will be explained in more detail later with reference to FIG. 2 and FIG. 3.

The rectal balloon 39 may include a valve 41 so that masses of intestinal gas can escape if the pressure increases. The valve 41 may open as soon as the intestinal pressure reaches a threshold value and may remain open until enough of the masses of gas have escaped for the excess rectal or intestinal pressure to have returned to normal. An excess pressure forming in the intestine may be equalized quickly. Equalizing the pressure in the intestine may cause the organ being examined to return to its original position so that, for example, an interrupted recording of measurement data may be resumed.

FIG. 2 shows a patient 43 positioned on a patient examination table 45 in an imaging device 51. The patient 43 is positioned in such a way that measurement data from the region being examined, in the illustrated case, the lower abdomen with the prostate, may be recorded. The imaging device 51 with a gantry 49 controlled by a computer unit 47 may be a nuclear medical imaging device suitable for PET or SPECT imaging. However, the imaging device 51 may be embodied as an MR device or CT device.

In one embodiment, the imaging device 51 is includes a C-arm having an X-ray detector on one end of the C-arm and an X-ray emitter on the opposite end of the C-arm. The imaging device 51 may be used for cone-beam CT.

As shown in FIG. 1, the sensor 37 has been rectally inserted into the intestine of the patient 43. The sensor 37 records a sensor signal that measures the pressure or pressure conditions in the intestine.

The sensor signal may be transmitted (fed) to the computer unit 47. The computer unit 47 may include an evaluation unit 53 that evaluates the sensor signal. The evaluation unit 53 may determine, for example, whether the measured pressure is within a predefined tolerance range or below a predefined threshold value. More complex evaluation algorithms can, though, also have been implemented in the evaluation unit 53.

The evaluated sensor signals or the measured and evaluated pressure may be forwarded to a recording unit 55. The imaging device 51, such as the gantry 49, may be controlled while the measurement data is being recorded based on the evaluated sensor signals or the measured and evaluated pressure. The recording unit 55 may have been implemented in the computer unit 47. The recording unit 55 may control the imaging device 51 in such a way that the recording of the measurement data is interrupted as soon as the measured pressure exceeds the threshold value or is outside the tolerance range.

The evaluated sensor signals or the measured and evaluated pressure may alternatively or additionally be forwarded to a processing unit 57. The processing unit 57 processes the recorded measurement data and reconstructs a medical image from the measurement data. The reconstruction of the medical image may depend on the various specific embodiments of the imaging device 51. For example, if the imaging device 51 is embodied for PET imaging, then the processing unit 57 analyzes the photons measured in the detectors while recording is in progress and from the measurement data reconstructs a spatially resolved and in particular three-dimensional image reflecting the distribution of the radioactive substance in the body.

The processing unit 57 may, during an image reconstruction, discard the measurement data when the measured pressure is outside a tolerance range or exceeds a threshold value. The reconstructed image may be stored in a memory or displayed to a user via a monitor 59.

FIG. 3 illustrates one embodiment of a method performed during the production of a medical image.

The recording 61 of measurement data is carried out with the aid of the imaging device. The pressure inside the body is measured simultaneously or almost simultaneously (pressure measurement 63) while the measurement data is being recorded.

An evaluation 65 of the measured pressure may be carried out during and/or after the recording of the measurement data. The measured pressure may be analyzed, for example, whether and when the measured pressure is within a tolerance range 67 or whether the measured pressure is below a threshold value 69.

The information obtained about the measured pressure may be used to induce an interruption 71 to the recording of measurement data for as long as the measured pressure corresponds to non-desired values. The information obtained as a result of the pressure measurement will affect the recording 61 of the measurement data.

The information obtained through pressure measurement may alternatively or additionally be used during a reconstruction 73 of the image carried out after the data recording to influence the reconstruction 73. For example, specific measurement data may be discarded if the measured pressure corresponded to non-desired values while said measurement data was being recorded. As an alternative thereto it is possible also to correct the measurement data by, for example, weighting it with other weighting factors during the image reconstruction.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for producing a medical image of a patient, the method comprising:
measuring a pressure inside a body of the patient over a period of time during a medical examination,
recording image measurement data originating in the patient during the examination, and
reconstructing a medical image from the recorded measurement data,
wherein recording of the measurement data is controlled with the measured pressure, reconstructing the medical image from the recorded measurement data is carried out using the measured pressure, or a combination thereof.

2. The method as claimed in claim 1, further comprising: evaluating the measured pressure during the examination, and interrupting the recording of measurement data when the measured pressure is outside a tolerance range or exceeds a threshold value.

3. The method as claimed in claim 1, further comprising: discarding a part of the recorded measurement data during the reconstruction of the medical image when the measured pressure is outside a tolerance range or exceeds a threshold value during the recording of said part of the measurement data.

4. The method as claimed in claim 1, wherein the medical imaging device is a nuclear medical imaging device, a magnetic resonance imaging device, or a computer tomography imaging device.

5. The method as claimed in claim 1, wherein the measured pressure inside the body is a gas pressure.

6. The method as claimed in claim 1, wherein measuring the pressure includes measuring a pressure in a hollow organ of the body.

7. The method as claimed in claim 6, wherein the pressure in the intestine is measured.

8. An imaging device for examining a patient, comprising:
a sensor that is operable to measure a pressure inside the body of a patient while measurement data is being recorded,
a recording unit that is operable to record the measurement data originating in the patient,
a processing unit that is operable to generate a medical image based on the recorded measurement data,
wherein the recording unit controls the recording of the measurement data based on the measured pressure, the processing unit reconstructs the medical image from the recorded measurement data using the measured pressure, or a combination thereof.

9. The imaging device as claimed in claim 8, wherein the sensor is operable to measure pressure in a hollow organ of the body.

10. The imaging device as claimed in claim 9, wherein the sensor is operable to measure pressure in the intestine.

11. The imaging device as claimed in claim 8, wherein the sensor is disposed on a valve mechanism for releasing an excess pressure.

12. The imaging device as claimed in claim 8, wherein the sensor is disposed on an inflatable balloon.

13. The imaging device as claimed in claim 8, wherein the sensor is operable to be inserted into in the intestine rectally.

14. The imaging device as claimed in claim 8, wherein the sensor is operable to measure a gas pressure.
